(19) United States
(12) Patent Application Publication
(10) Pub. No.: US 2007/0155967 A1
(43) Pub. Date: Jul. 5, 2007

(54) Process for producing piperidine-2,6 diones heterocyclically substituted in the 3-position

(75) Inventors: Oswald Zimmer, Wuerselen (DE); Fritz Theil, Berlin (DE); Olga Groeger, Berlin (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal Gmbh, Aachen (DE)

(21) Appl. No.: 11/604,788

(22) Filed: Nov. 28, 2006

Related U.S. Application Data
(63) Continuation of application No. PCT/EP05/05576, filed on May 24, 2005.

(30) Foreign Application Priority Data
May 28, 2004 (DE) 10 2004 026 703.0

(51) Int. Cl. A61K 31/517; C07D 403/02

(52) U.S. Cl. 544/284; 546/199

(57) ABSTRACT

An improved process for producing piperidine-2,6-diones heterocyclically substituted at the 3-position in a minimal number of reaction stages by reacting a 2-aminobenzylamine with a 3-bromoglutarimide to obtain an amine of formula (IV): 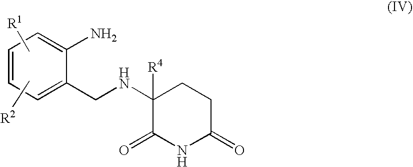 and thereafter reacting the obtained amine of formula (IV) with a suitable further reactant.

### CROSS REFERENCE TO RELATED APPLICATIONS

This application is a continuation of international patent application no. PCT/EP2005/005576, filed May 24, 2005 designating the United States of America, which was published in German on Dec. 8, 2005 as WO 2005/116008, the entire disclosure of which is incorporated herein by reference. Priority is claimed based on Federal Republic of Germany patent application no. DE 10 2004 026 703.0, filed May 28, 2004.

### BACKGROUND OF THE INVENTION

The present invention relates to a process for producing piperidine-2,6-diones heterocyclically substituted in position 3 corresponding to formula (I):

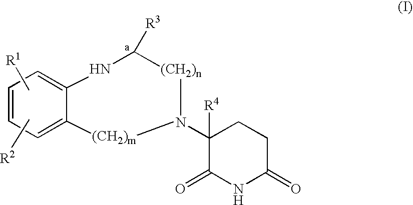

Piperidine-2,6-diones heterocyclically substituted in position 3, the production and use thereof in pharmaceutical preparations, in particular as immunomodulators for the treatment and/or inhibition of inflammatory and autoimmune diseases and of haematological/oncological disorders are known from published international patent application no. WO 03/053956 A1. The production processes described in this publication are in part very elaborate and require up to eight synthesis steps in order to obtain the target compound in the hydrochloride form. Accordingly, there has remained a need for a more efficient production process for such compounds.

### SUMMARY OF THE INVENTION

Accordingly, it is an object of the present invention to provide an improved process for producing piperidine-2,6-dione compounds heterocyclically substituted in the 3-position.

Another object of the invention was to provide a process for efficiently producing piperidine-2,6-dione compounds heterocyclically substituted in the 3-position in a reduced or minimal number of synthesis steps.

The present invention accordingly provides a process for producing piperidine-2,6-diones heterocyclically substituted in position 3 corresponding to formula (I): 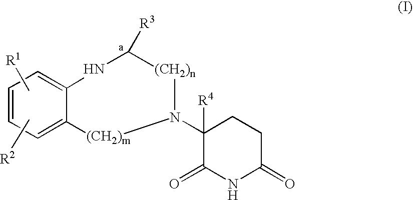
wherein
R¹ and R² may be the same or different and respectively represent H, Br, Cl, F, CF₃, OH, NO₂, NH₂, N(CH₃)₂, C₁₋₃ alkyl, C₁₋₃ alkoxy or phenyl, or together represent a fused benzene ring, wherein the rings are optionally substituted with at least one substituent selected from the group consisting of R¹ and R² as defined above;
if a represents a double bond, R³ represents hydrogen or methyl, or if a represents a single bond, R³ and the carbon atom to which it is attached form a carbonyl group;
R⁴ represents H, F, CF₃ or C₁₋₃ alkyl,
n is 0, and
m is 1;
   said process comprising:
   reacting a 2-aminobenzylamine of formula (II), 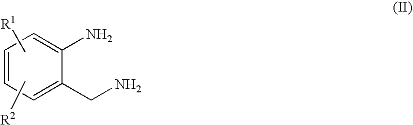
   wherein R¹ and R² have the above meanings,
   with a 3-bromoglutarimide of formula (III), 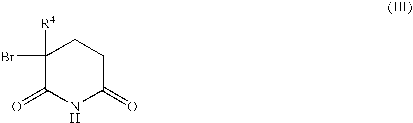
   wherein R⁴ has the above meaning,
   to yield an amine of formula (IV), 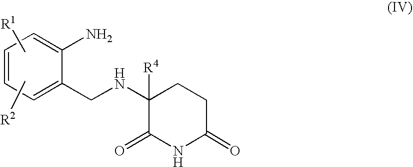
   and subsequently reacting the amine of formula (IV) with
(a) a compound of formula (V),
   R³—C(OR⁵ )₃ (V)
   wherein
R³ represents hydrogen or a methyl group, and
R⁵ denotes a linear or branched C₁-C₄ alkyl residue, or
(b) with an amidine salt of formula (VI), 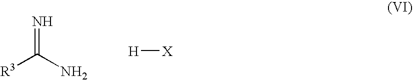
   wherein
R³ has the above meaning, and
X represents an anion of an acid, preferably acetic acid,
   to obtain a compound of formula I having a corresponding meaning of R³.

Compounds to be produced according to the invention corresponding to formula I, in which R³ together with the C atom to which it is attached represents a carbonyl group, are obtained by reaction of (IV) with C1 building blocks of formula (VII): 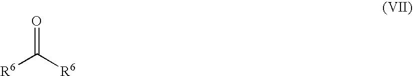
in which R⁶ denotes C1, the imidazol-1-yl residue, a C₁-C₄ alkoxy group, a phenyloxy group or a phenyloxy group substituted with a nitro group, chlorine or fluorine or a thiomethyl group, or by reaction of (IV) with C₁-C₄ alkyl esters, phenyl esters or substituted phenyl esters, preferably 4-nitro-, 4-chloro- or 4-fluorophenyl esters of chloroformic acid.

Compounds to be produced according to the invention corresponding to formula I, in which R³ together with the C atom to which it is attached represents a carbonyl group, are also obtained by reaction of (IV) with a C1 building block of formula (VIII):
C(OR⁷)₄ (VIII)
in which R⁷ represents a methyl or ethyl group.

In the process according to the invention, the reaction of the aminobenzylamines of formula (II) with the 3-bromoglutarimides of formula (III) preferably proceeds in inert solvents, preferably tetrahydrofuran or 1,4-dioxane, in the presence of tertiary amines, preferably triethyl- or ethyl-diisopropylamine at elevated temperature or in dimethylformamide as solvent at temperatures of between 0 and 100° C., preferably at 20° C.

Likewise preferably, the reaction of the compounds of formula (IV), which may optionally assume the form of salts, with the compounds of formula (V) is performed either without solvent or in an organic carboxylic acid, preferably acetic acid, in a temperature range of 10 to 150° C.

In comparison with the process known from WO 03/053956 A1 with its six stages and subsequent release of the base and hydrochloride precipitation, the process according to the invention makes it possible to achieve a highly advantageous 50% reduction in the synthesis steps.

The compounds produced according to the invention may be obtained as pure enantiomers or nonracemic enantiomer mixtures, racemates, diastereomers, or diastereomer mixtures either in the form of free bases or as salts with physiologically acceptable organic or inorganic acids. The product compounds have immunomodulatory activity, which means that they bring about a dramatic reduction in IL-12 production in LPS-activated monocytes with a simultaneous increase in IL-10 production. Based on this immunomodulatory activity, these compounds have great therapeutic potential in diseases in which pathogenesis has been attributed to excessive IL-12 production and a relative insufficiency of IL-10. In other words, these compounds are useful for treating and/or inhibiting inflammatory and autoimmune diseases. Due to the antiapoptotic action of IL-12, the compounds obtained according to the invention are also suitable for suppressing the formation of IL-12 in patients suffering from haematological and/or oncological disorders.

Inflammatory diseases which fall within the above-stated categories include inter alia inflammatory conditions of the skin (for example atopic dermatitis, psoriasis, eczemas, erythema nodosum leprosum), inflammatory conditions of the respiratory tract (for example bronchitis, pneumonia, bronchial asthma, ARDS (adult respiratory distress syndrome), sarcoidosis, silicosis/fibrosis), inflammatory conditions of the gastrointestinal tract (for example gastroduodenal ulcers, Crohn's disease, ulcerative colitis), together with diseases such as hepatitis, pancreatitis, appendicitis, peritonitis, nephritis, aphthosis, conjunctivitis, keratitis, uveitis, rhinitis. Autoimmune diseases include, for example, diseases of the arthritis category (for example rheumatoid arthritis, HLA-B27-associated diseases, rheumatoid spondylitis), together with multiple sclerosis, juvenile diabetes or lupus erythematosus. Further indications include sepsis, septic shock, bacterial meningitis, mycobacterial infections, opportunistic infections in AIDS, cachexia, transplant rejection reactions, graft-versus-host reactions, chronic cardiac failure, cardiac insufficiency, reperfusion syndrome and atherosclerosis, as well as chronic pain, fibromyalgia and reflex sympathetic dystrophy (RSD or Sudeck's disease). Clinical situations in which the immunomodulators produced in accordance with the invention can be used also include haematological diseases such as multiple myeloma, myelodysplastic syndrome and leukemias and further oncological diseases such as for example glioblastoma, prostate, kidney cell, mammary, thyroid, head and neck, pancreatic and colorectal carcinoma and melanoma and Kaposi's sarcoma.

### EXAMPLES

The invention will be explained in further detail hereinafter with reference to the following examples, which are solely for purposes of illustration and are not intended to limit the scope of the invention.

Silica gel 60 (0.040 to 0.063 mm) from E. Merck, Darmstadt, was used as the stationary phase for the chromatographic separations. The mixture ratios of the eluents are always stated in volume/volume.

The example produces were characterized by their melting point and/or NMR spectra. NMR spectra were recorded at 300 MHz using a Gemini 300 instrument from Varian. Chemical shifts are stated in ppm (6 scale). Tetramethylsilane (TMS) was used as the internal standard.

### Example 1

### 3-(4H-Quinazolin-3-yl)piperidine-2,6-dione

1st stage: 3-(2-Aminobenzylamino)piperidine-2,6-dione

A solution of 9.60 g of 3-bromopiperidine-2,6-dione [K. Fickentscher et al., Archiv der Pharmazie Weinheim, 1980, 481-487] in 120 ml of tetrahydrofuran was added dropwise over the course of 3 hours to a refluxing solution of 6.10 g of 2-aminomethylphenylamine and 14 ml triethylamine in 100 ml of tetrahydrofuran. Once addition was complete, the reaction mixture was stirred for a further 2.5 hours while being refluxed. The precipitated solid was filtered out, thoroughly washed with tetrahydrofuran and the combined filtrates were evaporated under a vacuum. The resultant crude product was purified by column chromatography on silica gel 60 (0.040-0.063 mm) from E. Merck, Darmstadt, with chloroform/methanol=10/1 as the eluent. 5.60 g (48% of theoretical) of the title compound were obtained in the form of crystals, which melted at 48 to 54° C.

2nd stage: 3-(4H-Quinazolin-3-yl)piperidine-2,6-dione

A mixture of 0.20 g of the product from stage 1 and 0.75 ml of orthoformic acid triethyl ester was stirred for 3 hours at 120° C. The reaction mixture was then evaporated under a vacuum, and the evaporation residue purified by flash chromatography on silica gel (c.f. stage 1) with ethyl acetate/methanol (2:1) as the eluent. 0.14 g (67% of theoretical) of the title compound were obtained in the form of virtually colorless crystals, which melted at 258° C.

¹H-NMR (300 MHz, DMSO-d₆): d=11.1 ppm (s, 1H), 7.1 (t, 1H), 7.1 (s, 1H), 7.0 (t, 1H), 6.95-6.85 (m, 2H), 4.55-4.25 (m, 3H), 2.85-2.55 (m, 2H), 2.7 (m, 1H), 2.0 (m, 1H).

### Example 2

### 3-(2-Oxo-1,4-dihydro-2H-quinazolin-3-yl)piperidine-2,6-dione

A solution of 2.20 g of the product from stage 1 of Example 1 and 1.78 g of N,N′-carbonyldiimidazole in 130 ml of tetrahydrofuran was stirred for 10 hours while being refluxed. After addition of a further 0.36 g of N,N′-carbonyldiimidazole, the mixture was stirred for a further 7 hours while being refluxed. The resultant solid was separated by filtration, washed with tetrahydrofuran, and dried under a vacuum. 1.50 g (62% of theoretical) of the title compound were obtained in the form of pale colored crystals.

¹³C-NMR (DMSO-d₆): 21.41; 31.19; 46.12; 55.28; 113.07; 117.81; 120.97; 125.23; 127.58; 137.02; 153.45; 170.83; 172.46.

### Example 3

### 3-(2-Methyl-4H-quinazolin-3-yl)piperidine-2,6-dione

A solution of 0.47 g of the product from stage 1 of Example 1 and 0.72 ml orthoacetic acid triethyl ester in 10 ml of acetic acid was stirred for 2 hours at 120° C. The reaction mixture was then evaporated under a vacuum and the residue purified by flash chromatography on silica gel with ethyl acetate/methanol (2:1). 0.44 g (86% of theoretical) of the title compound were obtained in the form of a greyish solid, which melted at 78 to 85° C.

¹H-NMR (DMSO-d₆): 1.92-2.00 (1H, m); 2.07 (3H, s); 2.36-2.47 (1H, m); 2.54-2.6 (1H, m); 2.78-2.89 (1H, m): 4,13 (1H, d, J=13, 8 Hz); 4.38 (1H, d, J=13, 8 Hz); 4,87 (1H, dd, J=12.8 and 5.0 Hz); 6.48 (1H, d, J=7, 7 Hz); 6.87-6.95 (2H, m); 7.08 (1H, dt, J=7.7 and 1.8 Hz); 10.98 (1H, br s).

¹³C-NMR (DMSO-d₆): 21.47; 21.96; 31.24; 43.47; 57.94; 121.62; 122.41; 123.49; 124.79; 127.46; 142.12; 157.12; 170.73; 172.44.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A process for producing a piperidine-2,6-dione heterocyclically substituted in the 3-position corresponding to formula (I): 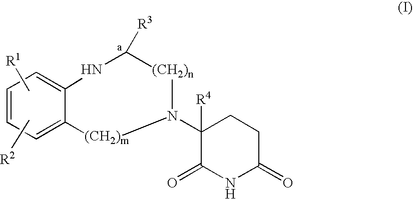
wherein
R¹ and R² may be the same or different and individually represent H, Br, Cl, F, CF₃, OH, NO₂, NH₂, N(CH₃)₂, C₁₋₃ alkyl, C₁₋₃ alkoxy, or phenyl or together form a fused benzene ring, wherein the rings are optionally substituted with at least one substituent selected from the group consisting of R¹ and R² as defined above;
if a represents a double bond, R³ denotes hydrogen or methyl, or
if a represents a single bond, R³ together with the carbon atom to which it is attached forms a carbonyl group;
R⁴ represents H, F, CF₃ or C₁₋₃ alkyl;
n is 0, and
m is 1,
said process comprising
reacting a 2-aminobenzylamine of formula (II): 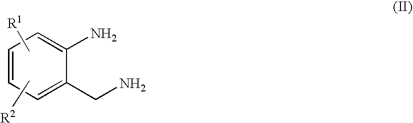
wherein R¹ and R² have the above meanings,
with a 3-bromoglutarimide of formula (III): 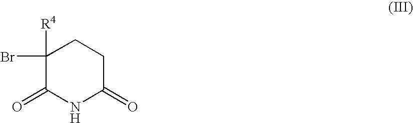
wherein R⁴ has the above meaning;
to obtain an amine of formula (IV): 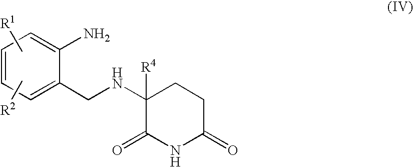
and thereafter reacting the obtained amine of formula (IV):
(a) with a compound of formula (V):
R³—C(OR⁵)₃ (V)
wherein
R³ represents hydrogen or a methyl group, and
R⁵ denotes a linear or branched C₁-C₄ alkyl residue; or
(b) with an amidine salt of formula (VI): 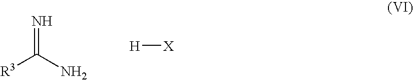
wherein
R³ represents hydrogen or a methyl group, and
X represents an anion of an acid; or
(c) with a C1 building block of formula (VII): 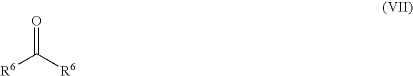
wherein R⁶ represents C1, the imidazol-1-yl residue, a C₁-C₄ alkoxy group, a phenyloxy group or a phenyloxy group substituted with a nitro group, chlorine or fluorine, or a thiomethyl group; or
(d) with a C₁-C₄ alkyl ester, phenyl ester or substituted phenyl ester of chloroformic acid, or
(e) with a C1 building block of formula (VIII):
C(OR⁷)₄ (VIII)
wherein R⁷ represents a methyl or ethyl group;
to obtain a corresponding compound of formula (I).

2. A process according to claim 1, wherein X represents an anion of acetic acid.

3. A process according to claim 1, wherein a compound of formula (IV) is reacted with a 4-nitro-, 4-chloro- or 4-fluorophenyl ester of chloroformic acid.

4. A process according to claim 1, wherein in the obtained compound of formula (I), R³ together with the carbon atom to which it is attached represents a carbonyl group.

5. A process according to claim 1, wherein an aminobenzylamine of formula (II) is reacted with a 3-bromoglutarimide of formula (III) in an inert solvent in the presence of a tertiary amine at elevated temperature.

6. A process according to claim 5, wherein the solvent is tetrahydrofuran or 1,4-dioxane.

7. A process according to claim 5, wherein said amine is triethylamine or ethyldiisopropylamine.

8. A process according to claim 1, wherein an aminobenzylamine of formula (II) is reacted with a 3-bromoglutarimide of formula (III) in dimethylformamide at a temperature of between 0 and 100° C.

9. A process according to claim 8, wherein the reaction is carried out at a temperature of about 20° C.

10. A process according to claim 1, wherein a compound of formula (IV) is reacted with a compound of formula (V) without solvent at a temperature of between 10 and 150° C.

11. A process according to claim 1, wherein a compound of formula (IV) is reacted with a compound of formula (V) in an organic carboxylic acid at a temperature of between 10 and 150° C.

12. A process according to claim 11, wherein the solvent is acetic acid.
